(19) United States
(12) Patent Application Publication
(10) Pub. No.: US 2009/0275919 A1
(43) Pub. Date: Nov. 5, 2009

(54) Balloon Deployment Device and Method

(75) Inventors: Joe Todd, Salt Lake City, UT (US); Neil Saunders, South Jordan, UT (US); David Wheeler, Salt Lake City, UT (US)

Correspondence Address: EDWARDS LIFESCIENCES CORPORATION, LEGAL DEPARTMENT, ONE EDWARDS WAY, IRVINE, CA, 92614, US

(73) Assignee: Edwards Lifesciences Corporation, Irvine, CA (US)

(21) Appl. No.: 12/434,190

(22) Filed: May 1, 2009

Related U.S. Application Data
(60) Provisional application No. 61/049,757, filed on May 1, 2008.

(51) Int. Cl. A61M 25/10

(52) U.S. Cl. 604/509; 604/103.01; 604/96.01

(57) ABSTRACT

Embodiments of the present invention provide systems and methods for deploying and/or retrieving a balloon configured to at least partially occlude a vessel, such as the coronary sinus. More particularly, embodiments of the present invention provide a stylet and a balloon catheter configured such that the stylet can be inserted into a lumen in the catheter and used to exert a force against an interior portion of the balloon in order to stretch the balloon and reduce the diameter of the balloon. In one embodiment, the stylet and the balloon both have specialty-configured tips that work together to prevent tearing of the balloon when the stylet is exerting a force against the interior of the balloon's tip.

### CROSS-REFERENCE TO RELATED APPLICATION

This application claims the benefit of U.S. Provisional Application No. 61/049,757, filed May 1, 2008, entitled “Balloon Deployment Device and Method”, which is also hereby incorporated herein by reference.

### FIELD

The invention generally relates to the field of balloon deployment devices and methods, and more particularly, embodiments of the present invention relate to a stylet configured for use in a balloon catheter to stretch the balloon so that it has a reduced diameter during insertion of the catheter into a vessel.

### BACKGROUND

Balloon catheters are catheters having balloon tips configured to at least partially occlude vessels, such as blood vessels, within the body. In some instances, the balloon catheters also deliver fluid to the vessel on one or both sides of the balloon. Furthermore, inflation of the balloon can help to anchor the catheter in a particular location within the vessel.

For example, balloon catheters are often used during heart surgery, where the patient is often placed on a cardiopulmonary bypass machine that performs the functions of the heart and lungs. While on cardiopulmonary bypass, blood flowing toward the heart and lungs is diverted to the bypass machine. The bypass machine oxygenates the blood and pumps it back into the patient to ensure the other organ systems receive sufficient oxygenated blood. The heart, however, is deprived of blood and, therefore, must be protected from damage. One conventional method of protecting the heart is to perfuse the myocardium with cardioplegic fluid. Temporarily arresting the heart using cardioplegic fluid and diverting blood flow away from the heart allows surgical operation on substantially bloodless and motionless heart chambers, surfaces, and related tributaries.

Cardioplegic fluid can be delivered to the myocardium in an antegrade or retrograde fashion. Antegrade delivery involves injecting the aorta or the coronary arteries directly with cardioplegic fluid so it travels along the path of normal blood flow to the heart muscle. Retrograde delivery involves injecting cardioplegic fluid into the coronary sinus, through which blood normally drains into the right atrium.

Often retrograde delivery is complimentary or preferable to antegrade delivery. Patients with significant obstruction in the coronary arteries (i.e., those undergoing coronary artery bypass surgery) may receive insufficient cardioplegia when it is delivered in an antegrade fashion. Patients with an incompetent aortic valve typically cannot receive cardioplegia in an antegrade fashion unless the aorta is opened and the cardioplegia is delivered directly to the coronary orifices. This results in longer operations and creates a risk of bleeding from an aortic suture line or damage to the coronaries themselves. Further, delivering cardioplegia in a retrograde fashion during some procedures, such as during replacement or repair of the aortic or mitral valves, protects the heart without interrupting the procedure to deliver cardioplegia in an antegrade fashion. Without these interruptions, cardiopulmonary bypass time and the duration of the cardiac arrest may be decreased.

Despite the potential advantages of retrograde delivery of cardioplegia, drawbacks in conventional catheter design often result in inefficient and/or ineffective delivery. One drawback of conventional catheters is that it is difficult to keep the catheter positioned in the sinus. Successful retrograde cardioplegia delivery requires that the distal end of the catheter remains securely positioned in the coronary sinus during fluid delivery. Typically, the catheter includes a balloon at the distal end of the catheter for securing the catheter in the coronary sinus so cardioplegic fluid can be repeatedly and accurately delivered. Such balloons, however, present other challenges of their own.

Specifically, delivering a balloon through the blood vessels and into the coronary sinus without damaging the blood vessels or the delicate coronary sinus can be a significant challenge. Damaging the coronary sinus or other blood vessels during surgery can create significant problems that can seriously complicate already complicated surgery. Furthermore, the balloon itself may also be fairly delicate. As a result, the devices and procedures used to deliver the balloon must be carefully configured so that they do not cause damage to the balloon. If the balloon is damaged during its delivery, the entire catheter will usually have to be replaced leading to increased surgery time and increased risk of complications resulting from inserting a second balloon catheter into the patient's vasculature. As such, there is a constant need for improved systems and methods for deploying a balloon in the coronary sinus during retrograde cardioplegia procedures or for deploying a balloon in other vessels for occluding the vessel and/or for delivering or retrieving fluid to or from the vessel.

### BRIEF SUMMARY OF EMBODIMENTS OF THE INVENTION

Embodiments of the present invention provide improved systems, devices, and methods for deploying a balloon within a vessel, such as a blood vessel. More particularly, embodiments of the present invention provide a balloon catheter having a balloon attached to its distal end. The catheter comprises a lumen extending therethrough, the lumen having a proximal opening and a distal opening. The distal opening of the lumen is surrounded by the balloon. Embodiments of the invention also provide a stylet having an elongate flexible wire and an enlarged tip at the distal end of the wire. The stylet can be inserted through the catheter's lumen and the stylet's tip pushed against the interior surface of the balloon to stretch and hold the balloon in a collapsed configuration during delivery and/or withdrawal of the balloon. Embodiments of the present invention further provide a stylet tip and a balloon tip that are specially configured to prevent tearing of the balloon and puncture of the vessel when the stylet is used to stretch the balloon.

More particularly, embodiments of the present invention provide a system for at least partially occluding a vessel in a patient. For example, in one embodiment, the system includes a catheter comprising a lumen having a proximal end, a distal end, and a diameter, and a balloon located at the distal end of the lumen. The system also includes a stylet comprising an elongate flexible wire having a proximal end, a distal end, and a diameter, and an enlarged tip coupled to the distal end of the wire. At least a portion of the stylet's enlarged tip has a diameter greater than the wire's diameter. The wire's diameter and the tip's greatest diameter are less than the lumen's diameter so that the wire and the tip are capable of being inserted through the lumen until the enlarged tip exerts a force against an interior surface of the balloon to reduce the diameter of the balloon.

In some embodiments, the enlarged tip has an elongate enlarged portion having a generally circular cross-section taken perpendicular to the wire's longitudinal axis. The diameter of the generally circular cross-section is greater than the wire's diameter and the enlarged tip is shaped such that increasingly more of the surface area of the interior of the balloon contacts the enlarged tip as the enlarged tip is increasingly pushed further into the interior surface of the balloon. In some instances, the diameter of the generally circular cross-section is approximately two times the wire's diameter. In one embodiment, the elongate enlarged portion is generally in the range of 0.3 to 1.0 inch long.

In one embodiment, the stylet's enlarged tip has a rounded distal end and, in some instances, has a semispherical portion and a generally cylindrical elongate portion extending proximally from the semispherical portion. The semispherical portion and the generally cylindrical elongate portion each have a diameter greater than the diameter of the elongate flexible wire. For example, in one embodiment, the diameter of the generally cylindrical elongate portion is approximately ⅛^{th} of an inch, while the diameter of the elongate flexible wire is approximately 1/16^{th} of an inch.

In some embodiments of the system the balloon includes a generally spherical portion and a nipple-like tip portion. In one exemplary embodiment, the distal end of the nipple-like tip portion has a radius of curvature that is equal to or substantially equal to the radius of curvature of the distal end of the stylet's enlarged tip. In this regard, the distal end of the nipple-like tip portion can have a semispherical curvature and a distal end of the stylet's enlarged tip can include at least a portion of a sphere. In one embodiment, the radius of semispherical curvature of the balloon's tip portion is approximately 0.015 inches larger than the radius of curvature of the distal end of the stylet's enlarged tip.

In an exemplary embodiment the nipple-like tip portion of the balloon includes at least one hole therein for allowing a perfusion fluid to pass therethrough. More generally, the balloon includes at least one hole therein for allowing perfusion fluid to pass therethrough and such a hole is typically located on a distal side of the balloon offset from the center of the distal side of the balloon. In general, the catheter further includes a second lumen for delivering perfusion fluid to the balloon and the catheter is configured such that the balloon is inflated with the perfusion fluid.

Embodiments of the present invention further provide a method of inserting a balloon catheter into a vessel, such as the coronary sinus. The method generally includes inserting a stylet into a lumen of the balloon catheter until a distal end of the stylet pushes against an interior surface of a balloon attached to the balloon catheter at the end of the lumen. The method also includes inserting the stylet into the lumen until the distal end of the stylet pushes a distal end of the balloon away from a proximal portion of the balloon and the diameter of the balloon is reduced.

Where the stylet comprises an elongate flexible wire and an enlarged tip at the distal end of the wire, the method typically also involves pushing the enlarged tip progressively further into the interior surface of the balloon such that progressively more of the interior surface of the balloon contacts the enlarged tip. In one embodiment, the method further involves inserting the balloon catheter with the stylet therein into a patient's blood vessel, such as the patient's coronary sinus. The method then involves removing the stylet and inflating the balloon after the balloon is properly located in the patient's blood vessel. Inflating the balloon sometimes includes the step of providing perfusion fluid into the balloon through a second lumen in the catheter. As described above, in some instances the balloon has at least one hole therein for allowing at least some of the perfusion flood to exit out of the balloon and into the patient's blood vessel.

Embodiments of the present invention further provide a stylet for use in a balloon catheter during insertion or withdrawal of the catheter into a vessel. Such a stylet generally includes a wire having a proximal end and a distal end, wherein at least the distal end of the wire is configured to be inserted into a lumen in the balloon catheter. The stylet also generally has an enlarged tip at the distal end of the wire, the enlarged tip being thicker than the wire, wherein the enlarged tip is configured to be pushed against an interior of a flexible balloon-shaped portion of the catheter to stretch or at least partially collapse the flexible balloon-shaped portion of the catheter.

In some embodiments, the stylet's enlarged tip is further configured such that, when the enlarged tip is pushed increasingly further into the interior surface of the flexible balloon-shaped portion of the catheter, progressively more of the interior surface of the flexible balloon-shaped portion of the catheter comes into contact with the enlarged tip. In this regard, in some embodiments, the enlarged tip includes a rounded distal end and a generally cylindrical portion extending from the distal end in the general direction of the proximal end of the wire.

### BRIEF DESCRIPTION OF THE SEVERAL VIEWS OF THE DRAWINGS

Having thus described embodiments of the invention in general terms, reference will now be made to the accompanying drawings, which are not necessarily draw to scale, and wherein:

FIG. 1 illustrates a balloon deployment system comprising a balloon catheter and a specially-configured stylet, in accordance with an embodiment of the present invention;

FIG. 2 illustrates how an enlarged tip of a stylet functions to stretch the balloon, in accordance with one embodiment of the present invention;

FIG. 3 illustrates how the enlarged tip of the specially-configured stylet of FIG. 1 functions to stretch the balloon without tearing the balloon, in accordance with another embodiment of the present invention; and

FIG. 4 illustrates using the catheter and the stylet of FIG. 1 to deploy a balloon in the coronary sinus, in accordance with an exemplary embodiment of the present invention.

### DETAILED DESCRIPTION OF EMBODIMENTS OF THE INVENTION

Embodiments of the present invention now will be described more fully hereinafter with reference to the accompanying drawings, in which some, but not all, embodiments of the invention are shown. Indeed, the invention may be embodied in many different forms and should not be construed as limited to the embodiments set forth herein; rather, these embodiments are provided so that this disclosure will satisfy applicable legal requirements. Like numbers refer to like elements throughout.

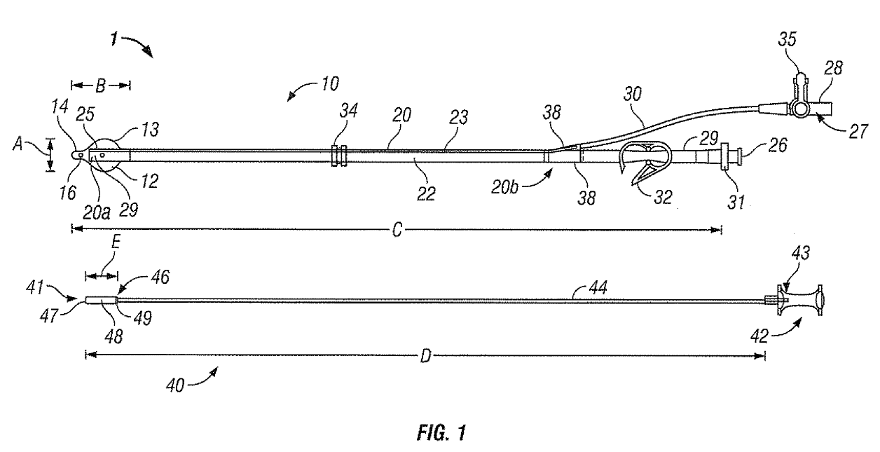

FIG. 1 illustrates a balloon deployment system 1 in accordance with an embodiment of the present invention. The balloon deployment system 1 generally includes a balloon catheter 10 and a stylet 40. The balloon catheter 10 is generally configured to deliver an expandable/collapsible balloon 12 attached thereto into a vessel, such as a blood vessel, so that the balloon 12, when expanded, can at least partially occlude the vessel. As described in greater detail below, the stylet 40 is configured to be inserted through a lumen in the catheter 10 and is specifically configured to be used for holding the balloon 12 in a collapsed state during insertion and/or withdrawal of the catheter 10 into and/or from the vessel, without damaging the balloon 12.

More particularly, the catheter 20 comprises an elongate cannula 20 having a distal end 20a and a proximal end 20b, with an expandable/collapsible balloon 12 extending from the catheter 20 at or near the distal end of the cannula 20. The cannula 20 comprises at least one lumen and, in some embodiments, comprises two or more lumens. In the illustrated embodiment, the cannula 20 is a dual-lumen cannula 20 having a first lumen 22 and a second lumen 23 extending along the length of the cannula 20. The first lumen 22 has a distal end 24 and a proximal end 26. The second lumen 23 also has a distal end 25 and a proximal end 27.

The cannula 20 must be sufficiently long and flexible to extend from the point where the catheter 10 is inserted into a patient's body to the point in the body that is to be occluded by the balloon. This can sometimes involve traveling through blood vessels and making one or more turns into one or more branches of the blood vessels. At the same time, the cannula 20 must be rigid enough so that it does not collapse or develop kinks unexpectedly when it is inserted into the patient. As such, in some embodiments, the cannula 20 has a generally tubular, wire-reinforced, silicone or polymeric structure suitable for introduction into a patient's body, as is generally known in the art. Furthermore, the diameter of the cannula 20 is sufficiently small to allow the cannula 20 to be inserted into the patient's blood vessel(s), such as the coronary sinus.

As described above, a balloon 12 extends from the catheter 10 at or near the distal end 20a of the cannula 20. In the illustrated embodiment the proximal end of the balloon 12 is attached to or otherwise extends from the side of the cannula 20 some distance from the distal end 20a of the cannula 20. As illustrated in FIG. 1, one embodiment of the balloon 12 has a generally spherical portion 13 and a nipple-like balloon tip portion 14 extending from the generally spherical portion 13. The diameter of the spherical portion 13 is generally configured to be equal to or greater than the diameter of the blood vessel to be occluded. As described below, there may be several advantages to the particular balloon shape illustrated in FIG. 1; however, other embodiments of the invention have a balloon 12 with a different shape. In this regard, in other embodiments of the invention the balloon 12 may be spherical, elliptical, cylindrical, barbell-shaped, or the like, with or without nipple-like tips extending therefrom.

In one embodiment, the balloon 12 is comprised of polyurethane. In other embodiments, however, the balloon 12 is comprised of other materials that are known in the art to be suitable for the particular application. In the illustrated embodiment, the external surface of the balloon 12 is generally smooth; however, in other embodiments, the external surface of the balloon 12 is rough or otherwise irregular to help prevent the balloon 12 from sliding within the vessel when the balloon is expanded. For example, U.S. Pat. No. 5,423,745 to Todd, which is assigned to the assignee of the present invention and which is incorporated herein by reference, describes balloons having irregular surfaces.

In the illustrated embodiment of the invention, in addition to occluding the vessel, the balloon catheter 10 is configured to deliver perfusion fluid, such as cardioplegia, to the vessel on the distal side of the occlusion. In this regard, the second lumen 23 is configured to deliver the perfusion fluid from outside the patient's body to the distal end 20a of the cannula. As such, the proximal end 27 of the second lumen 23 typically has a connector 28 structured to connect to a source of perfusion fluid. The source of the perfusion fluid may include, for example, a volumetric pump or a bag of solution with a pressure cuff. In some embodiments, the connector 28 includes a stopcock 35, clamp, or other device for controlling the flow of perfusion fluid through the second lumen 23.

In the illustrated embodiment, the balloon 12 is a “self-expanding” balloon that uses the pressure of the perfusion fluid flowing from the distal end 25 of the second lumen 23 to expand the balloon 12. More particularly, in the illustrated embodiment, the balloon 12 surrounds the distal end 25 of the second lumen 23 such that perfusion fluid exits the second lumen 23 into the interior of the balloon 12. The balloon 12 has one or more holes 16 on one side of the balloon 12 to allow the perfusion fluid to flow from inside the balloon 12 to the outside of the balloon and into the blood vessel in which the balloon 12 is located. The size of the hole(s) 16 in the balloon 12 and the pressure of the perfusion fluid in the second lumen 23 are selected such that the perfusion fluid creates enough pressure in the balloon 12 to cause the balloon 12 to expand sufficiently to occlude the blood vessel and/or to secure the catheter 12 in a certain position within the blood vessel.

In one embodiment, the one or more holes 16 are positioned in the tip portion 14 of the balloon 12. Furthermore, in one embodiment the holes 16 in the tip portion 14 of the balloon 12 are be specifically placed so that they are not located at the distal-most end of the tip portion 14 and are instead located in the sides of the tip portion 14. Such a location for the holes 16 decreases the risk of pushing the distal end 41 of the stylet 40 through the holes 16 when the stylet 40 is inserted into the catheter 10, as described in greater detail below.

In some embodiments, instead of a “self-expanding” balloon, the catheter 10 has a “manually-expanding” balloon at the distal end 20a of the cannula 20. In other words, the catheter 10 can comprise a balloon 12 that is expanded using a fluid other than the perfusion fluid or by some other device. For example, where the catheter 10 comprises a manually-expanding balloon, the catheter typically includes another lumen that is used to provide air or another fluid into the interior of the balloon for the purpose of inflating the balloon. Since the air or other fluid is used solely to inflate the balloon, the balloon generally does not include holes 16 as described above with regard to the self-expanding balloon 12 illustrated in FIG. 1. Instead, the cannula 20, or at least the second lumen 23, would extend through the manually-expanding balloon such that the opening in the cannula 20 or second lumen 23 is located distally from the balloon. In this way the cannula 20 or the second lumen 23 would be able to deliver perfusion fluid to the distal side of the balloon.

Referring again to FIG. 1, the distal end 24 of the first lumen 22, like the second lumen 23, opens into the inside of the balloon 12. As illustrated, in one embodiment, the proximal end 28 of the first lumen 22 includes a connector 31 having an opening therein for permitting the insertion of the distal end 41 of the stylet 40 into the first lumen 22. In some embodiments, the connector 31 comprises a seal, such as a fluid seal, that allows for passage of the stylet 40 but functions to prevent fluids from exiting or entering the first lumen 22 when the stylet 40 is inserted into the first lumen 22 and/or when the stylet 40 is removed from the first lumen 22.

As illustrated in FIG. 1, the distal portions of the first lumen 22 and second lumen 23 are integrally molded with the cannula portion 20 of the catheter 10. The proximal portions of the first lumen 22 and second lumen 23, however, are comprised of tubing 29 and 30, respectively, that extend from the proximal end 20b of the cannula 20. A stress relief sleeve 38 made of an elastic material surrounds the proximal end 20b of the cannula 20 and the distal ends of the tubing 29 and 30. Adhesive may be used to help adhere the stress relief sleeve 38 to the cannula 20 and to the tubing 29 and 30.

Since the tubing 29 and 30 typically remains outside the patient's body, the tubing 29 and 30 is generally not wire-reinforced like the cannula 20. The fact that the tubing is not reinforced may also permit the use of one or more clamps, such as clamp 32, on the tubing 29 and 30. As illustrated in FIG. 1, clamp 32 can be selectively used by a user of the catheter 10 to squeeze the tubing 29 to occlude the first lumen 22. A user of the catheter 10 may want to occlude the first lumen 22 to prevent fluid from exiting the first lumen 22 and/or to prevent anything from entering the first lumen 22. In some embodiments, the user may also use the clamp 32 or a similar clamp to squeeze the tubing 29 around the stylet's elongate wire 44 to lock the stylet 40 at a particular location within the first lumen 22

In some embodiments, the catheter 10 also includes a suture ring 34 mounted on the cannula portion 20 of the catheter 10. The suture ring 34 functions to aid in the attachment of the catheter 10 to the heart. In some embodiments of the present invention, the catheter includes a third lumen configured to be used for monitoring the pressure in the balloon 12 or in the blood vessel in the region of the balloon 12.

In order to insert the distal end of the catheter 10 into the patient and to be able to thread the catheter 10 through the blood vessels, the balloon 12 is collapsed to a diameter suitable for traveling through the blood vessels and for making necessary turns within the patient's vasculature. In this regard, as illustrated in FIG. 1, the balloon deployment system 1 further includes a stylet 40 that functions to collapse the balloon 12 by stretching the balloon 12 longitudinally along the axis of the stylet 40 such that the balloon's diameter is sufficiently reduced and so that the balloon 12 cannot freely move around the distal end of the catheter 10. More particularly, the stylet 40 is generally configured to be inserted into the catheter's lumen 20 until the distal end of the stylet 40 pushes against the interior of the balloon 12 so that balloon is stretched to a smaller diameter during delivery and/or recovery of the balloon 12 to and/or from a vessel.

In this regard, the stylet 40 generally comprises a handle 43 and an elongate wire 44 extending from the handle 43. The elongate wire 44 has a diameter smaller than the diameter of the first lumen 22. The length of the elongate wire 44 is generally at least slightly longer than the length of the first lumen 22. The elongate wire 44 is configured to be sufficiently flexible so that the wire 44 can bend around corners or other turns in the patient's vasculature; however, the wire 44, at the same time, is configured to be sufficiently rigid to allow a surgeon to use it for guiding the catheter through the patient's vasculature and to exert an axial force against the interior surface of the balloon 12. In an exemplary embodiment, the elongate wire 44 is made of 1/16^{th}-inch diameter stainless steel wire since this type of wire generally provides an appropriate combination of springiness and malleability.

In one embodiment of the invention, the distal end of the wire 44 is pushed directly against the interior surface of the balloon tip 14 in order to push the balloon tip 14 away from the distal end 20a of the cannula 20 to decrease the diameter of the spherical portion 13 of the balloon 12. Unfortunately, however, if the distal end of the wire 44 is too thin, the end of the wire 44 will easily and consistently puncture the tip 14 of the balloon 12 when the end of the wire 44 is pressed against the interior surface of the balloon 12. Besides destroying the balloon 12, if the stylet's tip punctures the balloon 12, then there is also a significant risk that the stylet's tip will puncture the coronary sinus or other blood vessel or organ surrounding the distal end of the catheter during deployment of the catheter into the patient.

The inventors of the present invention have determined that the stylet's tip generally needs to be approximately ⅛^{th} of an inch in diameter or greater to safely stretch the balloon 12 without significant risk of puncturing the balloon 12. However, the inventors of the present invention have also determined that a stylet 40 where the elongate wire 44 is made of a ⅛^{th}-inch diameter stainless steel wire is unacceptably stiff, at least for typical coronary sinus applications.

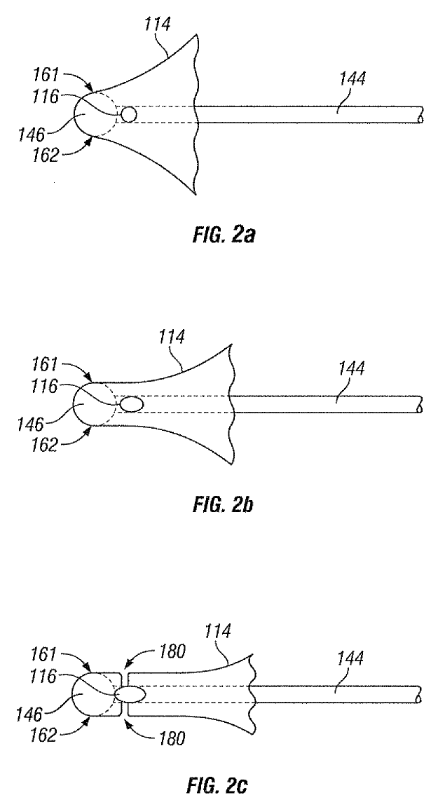

To solve this problem, embodiments of the present invention comprise a stylet 40 having an enlarged distal tip, where the enlarged distal tip has a diameter significantly greater than the diameter of the elongate wire 44. It is also preferable that the enlarged tip have a rounded end so that the end of the enlarged tip does not have sharp edges that can tear the balloon 12. As illustrated in FIG. 2, in one embodiment of the present invention the enlarged distal tip comprises a generally spherical tip 146 attached to a distal end of the elongate wire 144. As illustrated, the diameter of the generally spherical tip 146 is significantly greater than the diameter of the elongate wire 144. For example, in some embodiments the diameter of the spherical tip 146 is approximately double the diameter of the elongate wire 144. In one embodiment, an approximately ⅛^{th}-inch stainless steel ball 146 is attached to the distal end of an approximately 1/16^{th}-inch stainless steel elongate wire 144. In one embodiment, the generally spherical tip 146 has a diameter approximately equal to or just smaller than the diameter of the generally spherical curvature of the end of the balloon tip 114.

FIGS. 2a and 2b illustrate how the stylet's generally spherical tip 146 can be used to stretch and reduce the diameter of the balloon, in accordance with an exemplary embodiment of the present invention. For clarity, only the balloon tip 114 portion of the balloon is illustrated in FIG. 2, and the end of the cannula 20 is not depicted. FIG. 2a illustrates where the stylet has been inserted into the catheter to the point where the generally spherical tip 146 of the stylet just contacts the interior surface of the balloon tip 114 but exerts little or no force against the interior surface of the balloon tip 114. At this point, the balloon tip 114 and the remainder of the balloon are at rest and are not stretched. FIG. 2b illustrates where the stylet is pushed slightly further into the catheter so that it exerts a greater force against the interior surface of the balloon tip 114. This causes the balloon tip 114 of the generally flexible and somewhat resilient balloon to move away from the distal end of the cannula to which the proximal end of the balloon is attached. As such, the diameter of the balloon is decreased as the balloon tip 114 is pushed further away from the end of the cannula.

However, depending upon the strength of the balloon and the force necessary to reduce the diameter of the balloon, the generally spherical tip 146 may still tear the balloon. More particularly, in addition to the causing the balloon to have a reduced diameter, pushing the balloon tip 114 away from the distal end of the cannula also causes at least portions of the balloon to stretch. The inventors of the present invention determined that, as illustrated in FIG. 2c, if the stylet is pushed too far into the catheter, portions of the balloon may be stretched to the point of tearing 180. In some instances, the balloon may break before the balloon diameter is sufficiently reduced.

The inventors of the present invention also determined that, as illustrated in FIG. 2c, the balloon typically tears in the region located just proximal to the generally spherical tip 146. Specifically, the balloon material is typically stretched the most in the region located just proximal to the locations 161 and 162 on the balloon tip 114 where the balloon tip 114 first comes into contact with the stylet's generally spherical tip 146. Since, with the generally spherical tip 146, the locations 161 and 162 remain generally constant as the stylet is pushed further and further against the balloon tip 114, the region of the balloon located just behind the locations 161 and 162 becomes increasingly stretched until one or more tears 180 form in the region.

Therefore, to remedy this problem, preferred embodiments of the present invention provide a stylet tip and/or a balloon tip that are specifically configured to reduce the risk that the balloon will tear when the stylet is being used to stretch and reduce the diameter of the balloon. One exemplary embodiment of this type of preferred stylet tip and balloon tip is illustrated in FIG. 1. In the illustrated embodiment, the enlarged stylet tip 46 comprises a generally semispherical distal end 47, a generally cylindrical extended portion 48 extending proximally from the distal end 47, and an optional tapered portion 49. In the illustrated embodiment, the distal end 47 of the enlarged tip 46 is a hemisphere and the generally cylindrical extended portion 48 is a cylinder having the same diameter as the hemispherical distal end 47 and integrally formed with and extending from the hemispherical distal end 47. In other embodiments, however, the distal end 47 may have some other generally rounded shape and the generally cylindrical extended portion 48 may not be exactly cylindrical. For example, in one embodiment, the distal end 47 of the enlarged tip 46 comprises a semispherical tip that is less than a full hemisphere and the generally cylindrical extended portion 48 comprises a frustoconical shape having a generally circular cross-section that expands slightly in diameter as the extended portion 48 extends away from the tip's distal end 47. In another embodiment, the enlarged tip has a parabolic shape.

As illustrated in FIG. 1, the diameter of the generally semispherical distal tip 47 and the generally cylindrical extended portion 48 is significantly greater than the diameter of the elongate wire 44. For example, in some embodiments the diameter of the generally semispherical tip 47 and the generally cylindrical extended portion 48 is approximately double the diameter of the elongate wire 44. For example, in one embodiment, the diameter of the generally semispherical tip 47 and the generally cylindrical extended portion 48 is approximately ⅛^{th} of an inch, while the diameter of the wire 44 is approximately 1/16^{th} of an inch. In such an embodiment, the enlarged tip 46 may be, for example, 0.75 inches long, 0.6 inches long, or generally between approximately 0.3 inches long to 1.0 inches long. In one embodiment, the enlarged tip and the wire 44 are both comprised of stainless steel.

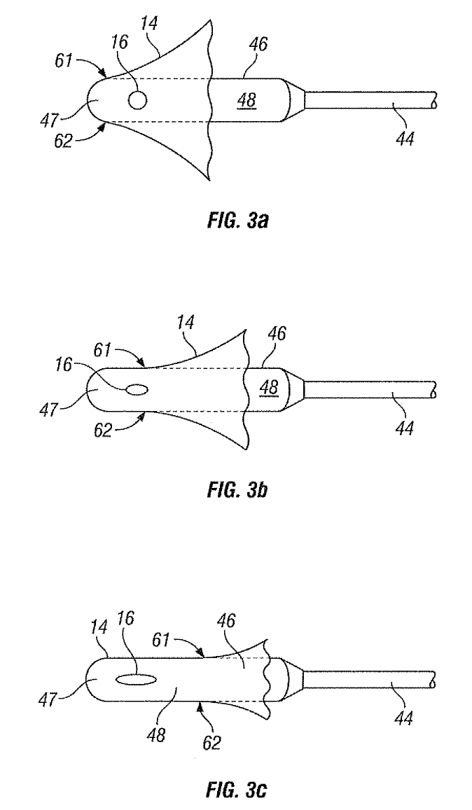

FIG. 3 illustrates how the embodiment of the stylet tip 46 and balloon tip 14 illustrated in FIG. 1 work together to reduce the risk of the balloon 12 tearing when the stylet 40 is used to reduce the diameter of the balloon 12. As described above, to reduce the diameter of the balloon 12, the enlarged stylet tip 46 located at the distal end 41 of the stylet 40 is pushed against the interior surface of the balloon tip 14 to stretch the balloon 12 axially, generally along the longitudinal axis of the catheter 10. For clarity, only the balloon tip 14 portion of the balloon 12 is illustrated in FIG. 3, and the end of the cannula 20 is not depicted.

FIG. 3a illustrates where the stylet 40 has been inserted into the catheter 10 to the point where the generally semispherical distal tip 47 of the stylet's tip just contacts the interior surface of the balloon tip 14, but exerts little or no force against the interior surface of the balloon tip 14. At this point, the balloon tip 14 and the rest of the balloon 12 are at rest and are not stretched. FIG. 3b illustrates where the stylet 40 is pushed slightly further into the catheter 10 so that the tip 46 of the stylet 40 exerts a greater force against the interior surface of the balloon tip 14. This causes the balloon tip 14 of the generally flexible and somewhat resilient balloon 12 to move away from the distal end of the cannula 20 to which the proximal end of the balloon 12 is attached. As such, the diameter of the balloon 12 is decreased as the balloon tip 14 is pushed further away from the end of the cannula 20. FIG. 3c illustrates where the stylet 40 is inserted into the catheter 10 such that the balloon 12 is fully stretched and reduced to a desirable diameter, in accordance with an embodiment of the invention.

Arrows 61 and 62 in FIG. 3 indicate locations on the balloon 12 where the balloon 12 first comes into contact with the surface of the enlarged stylet tip 46. Although, in FIG. 3, arrows 61 and 62 identify only two points on the balloon tip 14, it should be appreciated that this is due to the fact that the figures provides only a two-dimensional representation of the three dimensional balloon tip 14. As such, arrows 61 and 62 in reality refer more generally to a series of points or a line that surrounds the enlarged tip where the balloon 12 first comes into contact with the enlarged stylet tip 46.

As illustrated in FIG. 3, in this preferred embodiment, as the stylet's enlarged tip 46 is pushed further into the balloon tip 14, the locations 61 and 62 move along the surface of the enlarged tip 46 away from the tip's distal end 47. In other words, as the stylet's enlarged tip 46 is pushed increasingly further against the balloon tip 12, progressively more surface area of the balloon 12 comes into contact with the surface of the enlarged tip 46. Once the balloon 12 comes into contact with the enlarged stylet tip, friction between the two materials effectively retards further stretching in the area of contact. As such, the stretching of the balloon material occurs over a greater region of the balloon 12 and is not localized as described above with regard to the embodiment illustrated in FIG. 2. Therefore, the risk of tearing the balloon 12 is significantly reduced.

Therefore, it should be appreciated that, in preferred embodiments, the balloon 12 and the stylet's enlarged tip 46 have shapes and dimensions that are specifically configured to work together so that the locations 61 and 62 continually move proximally as the stylet tip 46 is pushed against the interior surface of the balloon 12 further or with greater force. In this regard, in one embodiment, the generally semispherical distal tip 47 has a diameter approximately equal to or just smaller than the diameter of the generally spherical curvature of the end of the balloon tip 14. In one embodiment, the balloon tip 14 starts out approximately 0.015 inches larger in diameter than the stylet tip 46 and gradually tapers out from there so that progressively more of the balloon tip 14 comes into contact with the stylet tip 46 as the balloon is stretched.

Furthermore, since friction between the balloon tip 14 and the stylet tip 46 is important to distribute the stretching over a greater area of the balloon 12, the balloon material, at least in the region of the balloon tip, and material at the surface of the stylet's enlarged tip are preferably selected so that there is a high degree of sliding friction between the two surfaces.

In some embodiments, the balloon 12 is completely flexible such that it becomes completely flaccid and collapses when the perfusion fluid or the other inflation fluid is not supplied to the balloon 12. In such embodiments, the stylet 40 is used to stretch the balloon 12 along the axis of the longitudinal axis defined by the cannula 20 to maintain the balloon in a low profile and to keep the balloon from flopping around in the blood vessel during delivery and/or retrieval. In other embodiments, however, the balloon 12 is semi-rigid such that it generally retains its “expanded” shape even when not inflated. In such embodiments, the stylet 40 is used to stretch the balloon 12 along the axis of the longitudinal axis defined by the cannula 20, thereby collapsing the balloon 12.

In some embodiments of the invention, the stylet 40 is configured to have a length such that, when the stylet is fully inserted into the catheter 10 the balloon 12 is stretched to a point where its diameter is sufficiently small to pass through the patients vasculature and where there is little risk of tearing or puncturing the balloon 12. In some embodiments, the length of the enlarged tip 46 is configured such that, when the balloon 12 is stretched the desired full amount, the proximal end of the enlarged tip 46 remains in the first lumen 22. In this way, there is less risk that the proximal end of the enlarged tip 46 will become hung up on the distal end of the cannula 20 when the stylet 40 is removed from the cannula 20. As described above, in some embodiments the proximal end of the enlarged tip 46 includes a tapered region 49. Such a tapered region 49 may be useful to also help reduce the risk that the proximal end of the enlarged tip 46 will become hung up on the distal end of the cannula 20 when the stylet 40 is removed from the cannula 20.

In one exemplary embodiment, the balloon deployment system 1 is specifically configured for performing retrograde perfusion of the coronary sinus. In this regard, in one exemplary embodiment of the system illustrated in FIG. 1, dimension A is approximately 0.58 inches, dimension B is approximately 1.08 inches, dimension C is approximately 12.31 inches, dimension D is approximately 12.88 inches, and dimension E is approximately 0.60 inches.

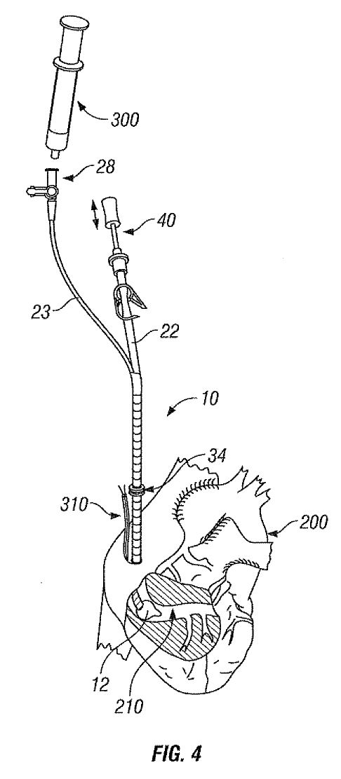

FIG. 4 illustrates how the balloon deployment system 1 can be used to occlude and provide retrograde perfusion of the coronary sinus, in accordance with one exemplary embodiment of the present invention. As illustrated in FIG. 4, the stylet 40 is inserted into the first lumen 22 of the catheter 10 until the distal end of the stylet exerts a force against the interior surface of the balloon 12, as described in greater detail above, thereby stretching and/or at least partially collapsing the balloon 12. An incision is made in the heart 200, typically in the right atrium. The stylet 40 is then inserted into the incision and the distal end of the catheter 10 is inserted into the coronary sinus 210 until the balloon 12 is properly positioned within the coronary sinus 210. A tourniquet 310 or other system is used in conjunction with the suture ring 34 to secure the catheter in relation to the heart 200. The stylet 40 is then removed from the catheter 10 or at least withdrawn from the balloon portion of the catheter 10. Perfusion fluid, such as cardioplegia, is then provided through the second lumen 23 from, for example, a plunger 300 or other source of perfusion fluid. This fluid inflates the balloon 12 to a desired pressure and secures the balloon's location within the coronary sinus 210.

Although the figures described above generally illustrate a catheter configured to both occlude and perfuse a vessel, in other embodiments of the present invention the catheter may only be configured to occlude, or partially occlude, a vessel. In still other embodiments, the catheter is configured to drain fluid on one or both sides of the balloon. In still other embodiments, the catheter is configured to supply two or more different fluids to the blood vessel and the catheter may use one or both of the fluids to expand the balloon. For example, in one embodiment, the catheter is configured to perfuse the vessel with two different fluids, one on each side of the occlusion. In such an embodiment, a third lumen is used to provide the second perfusion fluid and the third lumen has openings in the side of the cannula proximal to the balloon so that the second perfusion fluid perfuses the blood vessel on the proximal side of the occluding balloon. In other embodiments, however, the catheter has only one lumen and the same lumen is used for bother perfusion and insertion of the stylet.

In some embodiments of the present invention, the stylet 40 has other properties and components that assist with deployment of the balloon 12. For example, in one embodiment, the stylet 40 is configured such that the wire 44 assumes a predetermined curvature when the user actuates a trigger on the handle. This curvature may assist the user with locating and deploying the balloon in the coronary sinus. In this regard, U.S. Pat. No. 5,226,427 to Buckberg et al., which is assigned to the assignee of the present invention and which is incorporated herein by reference, describes an example of such a stylet.

Specific embodiments of the invention are described herein. Many modifications and other embodiments of the invention set forth herein will come to mind to one skilled in the art to which the invention pertains having the benefit of the teachings presented in the foregoing descriptions and the associated drawings. Therefore, it is to be understood that the invention is not to be limited to the specific embodiments disclosed and that modifications and other embodiments and combinations of embodiments are intended to be included within the scope of the appended claims. Although specific terms are employed herein, they are used in a generic and descriptive sense only and not for purposes of limitation.

What is claimed is:

1. A system for at least partially occluding a vessel in a patient, the system comprising:
a catheter comprising:
a lumen having a proximal end, a distal end, and a diameter, and
a balloon located at the distal end of the lumen; and
a stylet comprising:
an elongate flexible wire having a proximal end, a distal end, and a diameter; and
an enlarged tip coupled to the distal end of the wire, wherein at least a portion of
the enlarged tip has a diameter greater than the wire's diameter,
wherein the wire's diameter and the tip's greatest diameter are less than the lumen's diameter so that the wire and the tip are capable of being inserted through the lumen until the enlarged tip exerts a force against an interior surface of the balloon to reduce the diameter of the balloon.

2. The system of claim 1, wherein the enlarged tip has an elongate enlarged portion having a generally circular cross-section perpendicular to the wire's longitudinal axis, wherein the diameter of the generally circular cross-section is greater than the wire's diameter, and wherein the enlarged tip is shaped such that increasingly more of the surface area of the interior of the balloon contacts the enlarged tip as the enlarged tip is increasingly pushed further into the interior surface of the balloon.

3. The system of claim 2, wherein the diameter of the generally circular cross-section is approximately two times the wire's diameter.

4. The system of claim 2, wherein the elongate enlarged portion is in the range of 0.3 to 1.0 inch long.

5. The system of claim 1, wherein the enlarged tip comprises a rounded distal end.

6. The system of claim 1, wherein the enlarged tip comprises:
a semispherical portion; and
a generally cylindrical elongate portion extending proximally from the semispherical portion, wherein the semispherical portion and the generally cylindrical elongate portion each has a diameter greater than the diameter of the elongate flexible wire.

7. The system of claim 6, wherein the diameter of the generally cylindrical elongate portion is approximately ⅛^{th} of an inch, and wherein the diameter of the elongate flexible wire is approximately 1/16^{th} of an inch.

8. The system of claim 1, wherein the balloon comprises a generally spherical portion and a nipple-like tip portion.

9. The system of claim 8, wherein the distal end of the nipple-like tip portion comprises a radius of curvature that is equal to the radius of curvature of the distal end of the stylet's enlarged tip.

10. The system of claim 8, wherein the distal end of the nipple-like tip portion comprises a semispherical curvature, wherein a distal end of the enlarged tip comprises at least a portion of a sphere, and wherein the radius of semispherical curvature of the balloon's tip portion is approximately 0.015 inches larger than the radius of curvature of the distal end of the stylet's enlarged tip.

11. The system of claim 8, wherein the nipple-like tip portion of the balloon comprises at least one hole therein for allowing a perfusion fluid to pass therethrough.

12. The system of claim 1, wherein the generally spherical portion has a diameter of approximately 0.58 inches.

13. The system of claim 1, wherein the balloon comprises at least one hole therein for allowing perfusion fluid to pass therethrough, and wherein the hole is located on a distal side of the balloon offset from the center of the distal side of the balloon.

14. The system of claim 1, wherein the catheter further comprises a second lumen for delivering perfusion fluid to the balloon, wherein balloon comprises at least one hole therein for allowing perfusion fluid to pass therethrough, and wherein the catheter is configured such that the balloon is inflated with the perfusion fluid.

15. A method of inserting a balloon catheter into a vessel, the method comprising:
inserting a stylet into a lumen of the balloon catheter until a distal end of the stylet pushes against an interior surface of a balloon attached to the balloon catheter at the end of the lumen.

16. The method of claim 15, further comprising:
inserting the stylet into the lumen until the distal end of the stylet pushes a distal end of the balloon away from a proximal portion of the balloon and the diameter of the balloon is reduced.

17. The method of claim 16, wherein the stylet comprises an elongate flexible wire and an enlarged tip at the distal end of the wire, and wherein the method further comprises:
pushing the enlarged tip progressively her into the interior surface of the balloon such that progressively more of the interior surface of the balloon contacts the enlarged tip.

18. The method of claim 16, further comprising:
inserting the balloon catheter with the stylet therein into a patient's blood vessel.

19. The method of claim 18, wherein the blood vessel comprises the patient's coronary sinus.

20. The method of claim 18, further comprising:
removing the stylet and inflating the balloon after the balloon is properly located in the patient's blood vessel.

21. The method of claim 20, wherein inflating the balloon comprises:
providing perfusion fluid into the balloon through a second lumen in the catheter, wherein the balloon comprises at least one hole therein for allowing at least some of the perfusion flood to exit out of the balloon and into the patient's blood vessel.

22. A stylet for use in a balloon catheter during insertion or withdrawal of the catheter into a vessel, the stylet comprising:
a wire having a proximal end and a distal end, wherein at least the distal end of the wire is configured to be inserted into a lumen in the balloon catheter; and
an enlarged tip at the distal end of the wire, the enlarged tip being thicker than the wire, wherein the enlarged tip is configured to be pushed against an interior of a flexible balloon-shaped portion of the catheter to stretch or at least partially collapse the flexible balloon-shaped portion of the catheter.

23. The stylet of claim 22, wherein the enlarged tip is further configured such that, when the enlarged tip is pushed increasingly further into the interior surface of the flexible balloon-shaped portion of the catheter, progressively more of the interior surface of the flexible balloon-shaped portion of the catheter comes into contact with the enlarged tip.

24. The stylet of claim 22, wherein the enlarged tip comprises a rounded distal end and a generally cylindrical portion extending from the distal end in the general direction of the proximal end of the wire.
